(19) United States
(12) Patent Application Publication
(10) Pub. No.: US 2015/0025461 A1
(43) Pub. Date: Jan. 22, 2015

(54) Microfluidic Delivery Device

(71) Applicant: CorSolutions LLC, Groton, NY (US)

(72) Inventors: Thomas N. Corso, Groton, NY (US); Colleen K. Van Pelt, Groton, NY (US); Anthony J. Rose, Cortland, NY (US); Peter Weisz, Ithaca, NY (US)

(21) Appl. No.: 14/334,237

(22) Filed: Jul. 17, 2014

Related U.S. Application Data
(60) Provisional application No. 61/847,302, filed on Jul. 17, 2013.

(51) Int. Cl. F04B 49/22; A61M 39/24; A61M 5/142; F04B 53/00; F04B 19/00

(52) U.S. Cl. CPC F04B 49/22; F04B 53/001; F04B 19/006; A61M 5/142; A61M 39/24
USPC 604/151; 417/559; 417/540

(57) ABSTRACT

A system for the precise metering and delivery of discrete volumes is described. The system is composed of inexpensive components that can be easily assembled, allowing for cost-effective manufacturing of the system. The system can precisely meter and deliver fluids, solids in particulate or powder form, or alternatively it could mix discrete amounts of fluid and solid. Potential applications for the system and microfluidic device include subcutaneous, long-term, automated drug delivery, such as insulin for diabetics.

### CROSS REFERENCE

This application claims the benefit of the filing date of U.S. Provisional Patent Application Ser. No. 61/847,302, filed Jul. 17, 2013, which is hereby incorporated by reference in its entirety.

### FIELD

This invention relates to microfluidic delivery devices, systems, and methods utilizing such devices. In particular, this invention relates to microfluidic delivery devices useful as small volume, disposable medical devices for the precision delivery of medicines, drugs, or chemicals, such as insulin, and associated systems and methods.

### BACKGROUND

Insulin pumps are utilized by diabetics to automatically deliver insulin over extended periods of time. Most conventional insulin pumps employ the syringe mechanism as the fluid pumping means. With the syringe pump, the plunger of the syringe is advanced by a lead screw that is turned by a precision motor. The moving plunger forces fluid out of the syringe body and subsequently through a tube or catheter to the patient. The widespread use of syringe pump technology for fluid delivery in insulin pumps is mainly due to its ability to deliver the relatively small volume of insulin required by a typical diabetic in a continuous fashion. Typical insulin quantities delivered per day are in the regime of 0.1 to 1.0 milliliter. Additionally, a user may change the fluid delivery rate with a syringe pump and flow can be adjusted through a large range. By adjusting the motor rate, the user can change the amount of insulin delivered when needed. Although the syringe pump can deliver a liquid over a relatively wide range of flow rates, such performance comes at a cost. Currently available insulin pumps are complicated, expensive pieces of equipment costing thousands of dollars. This high cost is due primarily to the complexity of the stepper motor and lead screw mechanism. These components also contribute significantly to the overall size and weight of the insulin pump. Additionally, because of their cost, currently available insulin pumps have an intended period of use of up to two years, which necessitates routine maintenance of the device such as recharging the power supply and re-filling with insulin. These conventional insulin pumps also involve a large number of moving parts, and are mechanically complex. This not only increases size and weight, but also makes the manufacturing process of these insulin pumps very costly. Assembly is cumbersome and not advantageous for automated assembly. Due to the need for high tolerance parts and tedious, time-consuming manufacturing processes, skilled workers must manually assemble these insulin pumps by hand. The part and assembly costs for these pumps are exorbitant. The art lacks an insulin pump that can be easily assembled either manually or with automated equipment.

In addition to assembly and cost drawbacks, syringe pump technology is also not a continuous flow operating pumping mechanism. Once the syringe has expelled its volume, the syringe must be re-filled. Thus, the continuously expelled volume is limited by the syringe size. The larger the syringe, the poorer the fluid delivery resolution becomes. Therefore, smaller syringes are used, requiring frequent re-filling. The need to refill requires the use of additional valves and re-directing of fluid paths (i.e., reversing the plunger directly with no fluid re-routing mechanisms would pull fluid from the pump outlet), which in turn adds even more expense to the syringe approach. The art lacks a pumping mechanism which uses check valves for continuous pumping, and the use of a large fluid vessel to minimize the re-filling requirement which does not negatively affect the accuracy and precision of fluid being delivered.

For microfluidic applications, the re-filling of a syringe pump is not advantageous as a bubble may be introduced into the system. If valves are implemented to allow refilling from a vessel in the system, there is a stop in the pump's output during the refill process. Again, the art lacks a large fluid vessel to minimize the re-filling requirement without compromising accuracy and precision of the fluid delivery.

### SUMMARY

In accordance with one aspect of the present invention, there is provided a microfluidic delivery device for pumping a predetermined volume of fluid including a housing including a channel connecting a fluid inlet and a fluid outlet; a moveable member positioned in the channel between the fluid inlet and the fluid outlet, moveable between a fill position stop and a dispense position stop, where a positive pressure is created in the channel when the member moves to the dispense position stop and negative pressure is created in the channel when the member moves to the fill position stop; a cavity capable of accepting fluid from the channel when the moveable member moves from the dispense position stop to the fill position stop; an inlet check valve positioned between the fluid inlet and the moveable member such that an inlet check valve open position allows fluid flow in the direction from the fluid inlet to the moveable member and an inlet check valve closed position prevents fluid flow in the direction from the moveable member to the fluid inlet; and an outlet check valve positioned between the fluid outlet and the moveable member such that an outlet check valve open position allows fluid flow in the direction from the moveable member to the fluid outlet and an outlet check valve closed position prevents fluid flow in the direction from the fluid outlet to the moveable member, wherein the outlet check valve is in the closed position when fluid is drawn from the fluid inlet and through the open inlet check valve into the cavity when the moveable member is moved to the fill position stop and the inlet check valve is in the closed position when fluid is expelled from the cavity through the open outlet check valve and out the fluid outlet when the moveable member is moved to the dispense position stop.

In accordance with another aspect of the present invention, there is provided a microfluidic delivery device for pumping a predetermined volume of fluid including a housing having a channel connecting a fluid inlet and a fluid outlet; a moveable member in communication with the channel between the fluid inlet and the fluid outlet, moveable between a fill position stop and a dispense position stop, where a positive pressure is created in the channel when the member moves to the dispense position stop and a negative pressure is created in the channel when the member moves to the fill position stop; a cavity capable of accepting fluid from the channel when the moveable member moves from the dispense position stop to the fill position stop; an inlet check valve positioned in the channel between the fluid inlet and the moveable member such that an inlet check valve open position allows fluid flow in the direction from the fluid inlet to the cavity and an inlet check valve closed position prevents fluid flow out the fluid inlet; an outlet check valve positioned in the channel between the fluid outlet and the movable member such that an outlet check valve open position allows fluid flow in the direction from the cavity to the fluid outlet and an outlet check valve closed position prevents fluid flow in the direction from the fluid outlet to the inlet check valve; and an isolation feature in communication with the cavity, wherein the outlet check valve is in the closed position when fluid is drawn from the fluid inlet and through the open inlet check valve into the cavity when the moveable member is moved to the fill position stop and the inlet check valve is in the closed position when fluid is expelled from the cavity through the open outlet check valve and out the fluid outlet when the moveable member is moved to the dispense position stop.

In accordance with another aspect of the present invention, there is provided a method for pumping a predetermined volume of fluid by utilizing the present microfluidic delivery device.

In accordance with another aspect of the present invention, there is provided a method for administering a predetermined volume of medicine to a patient by utilizing the present microfluidic delivery device.

These and other aspects of the present invention will become apparent upon a review of the following detailed description and the claims appended thereto.

### BRIEF DESCRIPTION OF THE DRAWINGS

FIG. 1A is a schematic of an embodiment of the microfluidic delivery device of the present invention where two check valves are each connected to an inlet and an outlet through a fluid conduit with the moveable member shown in the dispense position stop;

FIG. 1B is a schematic of the microfluidic delivery device where two check valves are each connected to an inlet and an outlet through a fluid conduit with the moveable member shown in the fill position stop;

FIG. 2A is a schematic of an embodiment of the microfluidic delivery device where two check valves are in series with each other with the moveable member shown moved to the fill position stop;

FIG. 2B is a schematic of the microfluidic delivery device where two check valves are in series with each other with the moveable member shown moved to the dispense position stop;

FIG. 3 is a schematic of the microfluidic delivery device showing the moving member connected through drive components, including a rotational mechanism and return force springs of the check valves;

FIG. 4 is a perspective exploding view showing a cross-section of the main block with two end caps; and

FIG. 5 is a schematic of the microfluidic delivery device housed in a protective enclosure with a power source, drive mechanism, motor, and delivery needle and/or catheter, or outlet conduit.

### DETAILED DESCRIPTION

A delivery system constructed according to the present invention can be utilized in a variety of applications. The invention may, for example, deliver liquid to a body or micro-device. In addition to delivering flow downstream from the fluid outlet through positive pressure, another use includes pulling liquid via negative pressure through a fluidic device upstream from the fluid inlet. One such application is for the delivery of medication, drug, or chemical to a person or animal. The invention can be applied in other medical fields, such as for implantable micro-pump applications, or in non-medical fields such as for small, low-power, precision lubricating pumps for precision self-lubricating machinery. Other areas of use include nanotechnology and microtechnology, such as Lab-on-a-Chip, BioMEMS, and Point-of-Care Devices.

The present invention provides a microfluidic delivery device useful as a mechanical drug and/or insulin delivery device for diabetics that avoid the limitations of the syringe pump, such as size, weight, cost, complexity, and assembly requirements. The present invention dramatically simplifies the manufacturing process as compared to those required by syringe pumps. Importantly, it also avoids the dependency of the fluid delivery accuracy and precision on the syringe volume (fluid holding capacity). By overcoming these limitations, in an embodiment, a precise and reliable insulin delivery system can be produced with sufficiently low cost while being small in size and low in weight for easy portability by the user. Such a device may be worn discretely on the skin as an adhesive patch and contain a multi-day supply of insulin after the use of which the device is disposed of and replaced. Alternatively, since the device can function in the same manner as a continuous flow device without the requirement of stopping flow to re-fill, it may also serve as a long-term uninterrupted pump.

The present invention incorporates a miniature dual check-valve system which allows a pre-determined volume or aliquot of medicine, drug or chemical in fluid or solid particulate form, to be introduced into a secondary system, such as a body or fluid receiving device. The present invention is designed to supply periodic dosing by providing sequential defined volumes of fluids. In accordance with the present invention, the fluid or solid particulate is delivered in periodic discrete doses of a small fixed volume rather than in a continuous manner. The overall liquid delivery rate of the device is controlled by adjusting the dosing frequency. The device utilizes a precision timing mechanism along with a simple mechanical system. The method and invention result in device that is small in size, simple, and amenable to simple production processes and automation.

It is an underlying assumption of the invention that in the treatment of, for example diabetes, there is no clinical difference between administering insulin in periodic discrete small doses and administering insulin in a continuous flow, as long as the administration period of the discrete dose is small compared to the interval of time between which the blood glucose level is measured. For the present invention, a small dose size is regarded as on the order of 0.10 units of insulin (1 microliter) assuming a standard pharmaceutical insulin preparation of 100 units of insulin per milliliter. A typical insulin dependent diabetic person uses between 10 and 100 units of insulin per day, with the average diabetic person using about 40 units of insulin per day. Thus, the present invention is capable of delivering the daily insulin requirements of the average diabetic person in 400 individual discrete doses of 1 microliter each with a dosing period that can be programmed by the user. A pump constructed according to the present invention can have a predetermined discrete dosage volume that is larger or smaller than 1 microliter, but preferably falls within the range of 0.5 to 5 microliters. The smaller the discreet dose, the more energy required by the device to deliver the given amount of fluid, since each pump cycle consumes roughly the same amount of energy regardless of discrete dosage size. On the other hand, the larger the discrete dosage is, the less precisely the pump can mimic the body in providing a smooth delivery rate. The amount of fluid delivered in each pump cycle is specific to the pump design. A device constructed according to the present invention is also suitable for delivery of other medicines, drugs or chemicals. In an embodiment, the discrete doses may be delivered to an additional function component, such as a dampener or restrictive feature, to smooth the flow, achieving a fluid delivery profile similar to that of continuous flow.

It is further intended that the present invention could be used as a disposable component of a larger diabetes management system composed of additional disposable and non-disposable components.

The device in accordance with the present invention may serve as a delivery device for applications used in microfluidics; lab-on-a-chip; cells-on-a-chip; body-on-chip; a delivery for 3D or flow cell culture; bioMEMs; sensors and mechanical devices requiring a fluid source; precision metering & mixing; flow chemistry: pump for delivery reactants, solutions, fluid manipulation; PCR: fluid delivery; DNA sequencing: for manipulation of the DNA in a fluid; miniature detectors: spectroscopy, mass spectrometry, detectors for separation devices; and particle manipulation.

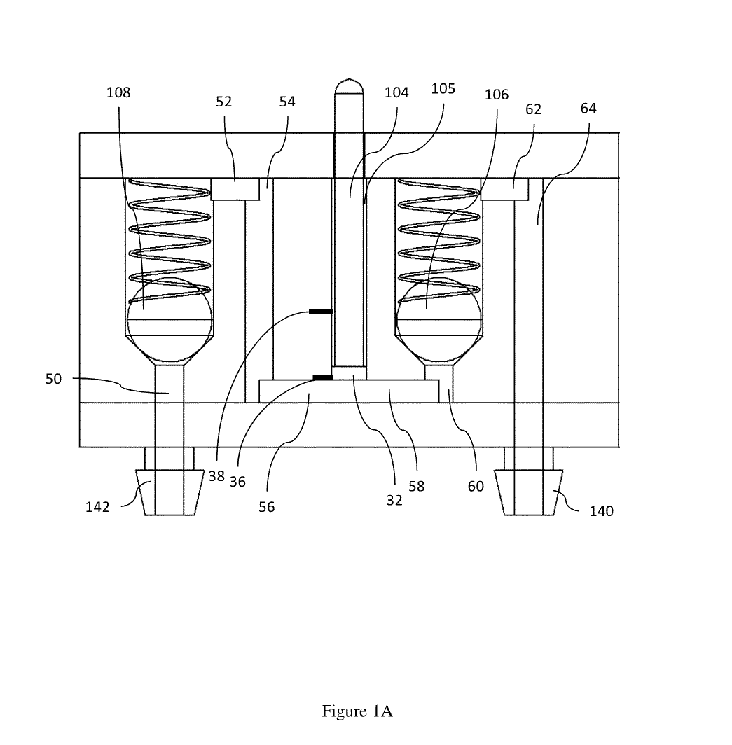

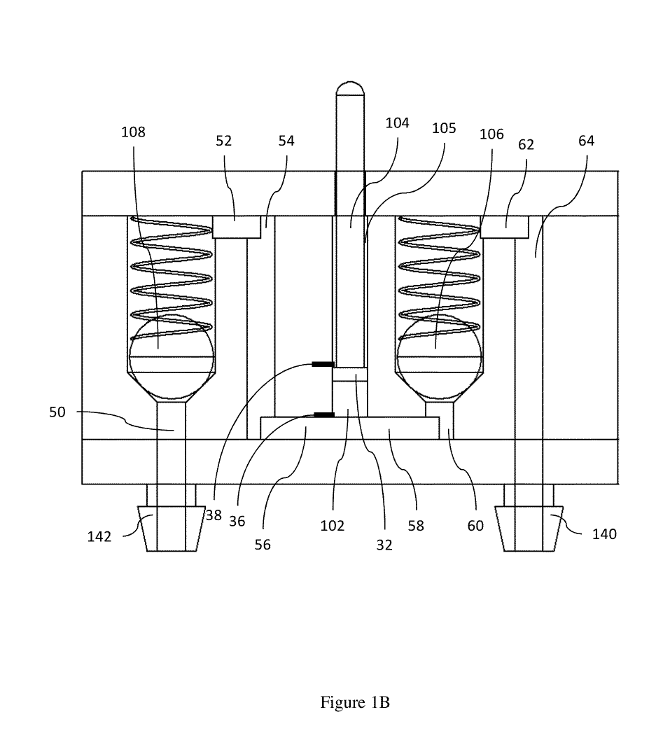

In an embodiment a fluid inlet 142, a fluid outlet 140, inlet check valve 108, outlet check valve 106, a moving member 104 and seal 32, a moving member holder 105, and a conduit, shown as connecting channels 50, 52, 54, 56, 58, 60, 62 and 64 of the device is shown in FIG. 1. Although the device has been described and shown in the figures as having a plurality of channels for clarity of description, the device can also be described in terms of having a single continuous channel. Prior to use, the device is primed and purged so that all fluid channels 50, 52, 54, 56, 58, 60, 62, 64 and check valves 106, 108 are filled with fluid and the fluid path of the device is devoid of any air. Fluid is manipulated in the device to achieve dispensing of a precise, pre-determined fluid volume, which can be repeated multiple times. To accomplish this, the moving member 104, which has a seal 32, displaces liquid by creating either a positive or negative pressure within the channel. The displacement occurs when the moving member 104 is alternated between a dispense position stop 36 (as shown in FIG. 1A) and a fill position stop 38 (as shown in FIG. 1B). When the moving member 104 is moved from the dispense position stop 36 to the fill position stop 38, negative pressure is created in the device and fluid is drawn from the intake channel. This negative pressure causes fluid, for example, contained in a reservoir connected to the fluid inlet 142, to be aspirated into the device. Fluid enters at the fluid inlet 142, and fluid travels through channel 50, through the inlet check valve 108 which is in the open position, and through connecting channels 52, 54 and 56, filling the cavity 102 (FIG. 1B) created by the withdrawing moving member 104. When filling, the fluid has minimal resistance to fill the cavity 102 since inlet check valve 108 is in the open position. However, outlet check valve 106 is in the closed position so no fluid or air is drawn in from the fluid outlet 140 or connecting channels. Therefore, when in the fill position stop 38, no fluid is dispensed from the device. When the moving member 104 is moved from the fill position stop 38 to the dispense position stop 36, positive pressure is created and the fluid will follow the path of least resistance, which is through connecting channels 58 and 60, through open outlet check valve 106, through connecting channels 62 and 64, and through the fluid outlet 140. Inlet check valve 108 is in the closed position preventing fluid leakage through the inlet. Thus, the device accurately and precisely measures the volume of fluid or metered aliquot, contained in the cavity 102 created by the moving member 104 when moving from the dispense position stop 36 to the fill position stop 38. A check valve is capable of one-way fluid flow operation only. Ball check valves are shown in the figures, however, any type check valves are suitable for use in the present invention. Optionally, the check valves 106 and 108 are equipped with springs to maintain the ball check valves in a closed position when there is no fluid movement through the system, for example, when the moving member 104 is at rest.

The moving member 104 can be driven, for example, in a reciprocating motion using one or more hard stops, such as the fill stop and the dispense stop, as described here. Using hard stops, a controlled stroke between the stop positions is achieved. This allows for a precise, pre-determined fluid volume or metered aliquot to be dispensed from the device. The pre-determined volume of the metered aliquot can be varied as desired by adjusting the distance between the stops. In an embodiment, as shown in FIGS. 1A and 1B, the fill position stop 38 is mechanical in nature, as the stop 38 extends into the moving member holder 105. As the moving member 104 moves toward the fill position, seal 32 on the moving member contacts the fill position stop 38, preventing the moving member from extending further. The seal 32 contacts and seals against the inner wall of the moving member holder 105, preventing fluid from extending past the fill position 38. Alternatively, stops for the moving member could be located anywhere along the moving member 104 or moving member holder 105; or integrated with the moving member drive mechanism. Movement of the moving member may be controlled as known in the art. For example, the stop for the fill position 38 could be mechanical or sensor-based.

A stop for the dispense position stop 36 could also be mechanical or sensor-based. A mechanical stop is shown (FIG. 1A) where the seal 32 on the end of the moving member 104 is larger than the opening to the connecting channels 56, 58. Thus, when the moving member is moved to the dispense position stop 36, the seal 32 contacts the channel wall, preventing the moving member from further advancement. An alternative stop for the dispense position stop 36 can also be located at the end of the moving member 104, effectively extending the length of the moving member 104. When moved into the dispense position, this stop feature enters the connecting channel 56, 58 and contacts with the opposite wall of the connecting channel 56, 58. This stop feature prevents the moving member 104 from extending beyond the dispense position stop 36. Such dispense position stop allows for the cavity 102 bottom to be adjacent to a channel which minimizes dead volume.

The sealing surface to the cavity may be molded on the moving member 104 or be a separate component seal 32 that integrates onto the moving member. The moving member may have a feature at the cavity end that can limit the forward movement of the member. Seal 32 may also function to set the distance based on its length. Alternatively, the moving member 104 could have a plurality of accurate position control stops, which would allow a continuum of volumes to be dispensed.

The fill and dispense positions may be controlled through physical stops or position sensors. Connecting channels may have various shapes, surfaces, and pathways depending on the desired fluidic path and behavior. Surfaces may be native or tuned or modified to be hydrophilic or hydrophobic or a mix thereof, in order to affect wetting properties of a given fluid. A bubble trap may be incorporated in the fluidic pathway if desired. Many check valve types and mechanisms may be employed and are known to those skilled in the art.

As medicines, such as insulin, often require specific storage requirements, the invention has been designed so that the device can be manufactured and delivered to the end-user without the insulin in situ. Once the device is ready to be employed, a user may insert an insulin filled reservoir at the fluid inlet 142 site of the device. The fluid reservoir containing medicine, such as insulin, can have a collapsible feature. The collapsible nature of the reservoir would ensure that vacuum lock or pressure build-up which could create air pockets that would interrupt fluid delivery will be minimized or avoided. The fluid reservoir may also have one or more air bleeding components. For a rigid or semi rigid reservoir, the reservoir may have a moving component allowing for the reservoir to hold the insulin while not building significant negative pressure to inhibit withdrawal of the insulin. If refilling of the reservoir is desirable, one or more fill ports could be placed at any location on the fluid reservoir.

The device is able to be primed prior to use to evacuate any gas or air from the system and to fill the system with the desired fluid. For precision fluid delivery devices, priming the device to eliminate any air can be important. Air left remaining within the device due to insufficient priming, may compromise the fluid delivery precision of the device. Alternatively, medicines, such as insulin, could be incorporated into the invention at the time of manufacture, preventing the need for the user to prime the system prior to use.

In the embodiment shown in FIG. 1, prior to use, one could prime the device by filling all paths with fluid. Fluid can be easily swept from the fluid inlet 142, all the way through the device, to the fluid outlet 140. This can be accomplished in a number of ways. Firstly, the repeated motion of the moving member 104 between the fill position stop 38 and the dispense position stop 36, will allow for fluid to fill the fluid inlet 142, channel, inlet check valve 108, outlet check valve 106 and fill the fluid outlet 140. Another way of priming would be by providing a flow from an external source with sufficient pressure and volume connected to the fluid inlet or outlet or both to fill the device with fluid. Either way would prime the device by filling all paths with fluid and voiding the device of air. Further, a user could prime the device by injecting fluid (insulin) into the device. This efficient design allows the swept volume to be in all one path, as shown in FIG. 1.

In the configuration shown in FIGS. 1A and 1B, the moving member 104 is positioned in the channels between both check valves and the fluid inlet and fluid outlet. The inlet check valve 108 is positioned between the fluid inlet 142 and the moving member 104 such that the open position allows fluid flow in the direction from the inlet to the moving member and the closed position prevents fluid flow in the direction from the moving member to the inlet. The outlet check valve 106 is positioned between the outlet 140 and the moving member 104 such that the open position allows fluid flow in the direction from the moving member to the outlet and the closed position prevents fluid flow in the direction from the outlet to the moving member. As shown in FIG. 1A, the seal 32 is preferably flush or near flush with the top of the channel 56, 58 when the moving member 104 is in the dispense position stop and the seal 32 forms a sealing surface at the dispense position stop 36 which is at the top of the channel 56, 58. The sealing surface effectively becomes the outer wall of the channel 56, 58. This design results in a flow path without edges or angles and is optimal for sweeping out bubbles with the liquid priming step.

Alternately, if the device or its fluidic components are under vacuum prior to filling with fluid, the device will readily aspirate the fluid, such as insulin. This would serve to prime the device, eliminating any air from the fluid path. The entire device or selected components of the device could be placed and stored under vacuum at the time of manufacture. Alternatively, the channels could be pre-evacuated or the vacuum accomplished at the time of use with a vacuum system method.

Following the priming of the device, when there is no fluid movement, both check valves are closed. To operate the device, the moving member 104 is moved from the dispense position stop 36 into the fill position stop 38, creating a cavity 102, as shown in FIG. 1B. The negative pressure introduced to the intake portion of the system by the withdrawal of the moving member causes fluid to enter the device from the fluid inlet 142, travel through channel 50, through open inlet check valve 108, through channels 52, 54 and 56, and to the cavity 102. The cavity 102 will fill with liquid. The negative pressure introduced reinforces the closed position of outlet check valve 106. When the cavity 102 is filled with fluid, fluid movement stops and the inlet check valve 108 closes. To dispense fluid, the moving member 104 is moved to the dispense position stop 36 creating positive pressure introduced to the dispense portion of the system. The positive pressure reinforces the closed inlet check valve 108 and forces the volume of fluid held in the cavity 102 to force fluid through open outlet check valve 106 and exit the fluid outlet 140. Thus, a precise amount or metered aliquot of fluid is dispensed with each intake and dispense cycle. This procedure can be automated to produce a plurality of dispensing cycles of fluid.

The invention preferably includes an on-board diagnostic that detects movement or position of the displacement mechanism or the mechanical drive system. The diagnostic could contain position sensors or motion sensors. The sensing may be electrical or mechanical based or a combination of both. Alternatively, the invention could use an inline flow sensor to measure flow rate. In an embodiment, the metered aliquot can be adjusted to a desired volume to set a desired dosing level. The locations of the fill stop and dispense stop can be adjusted accordingly.

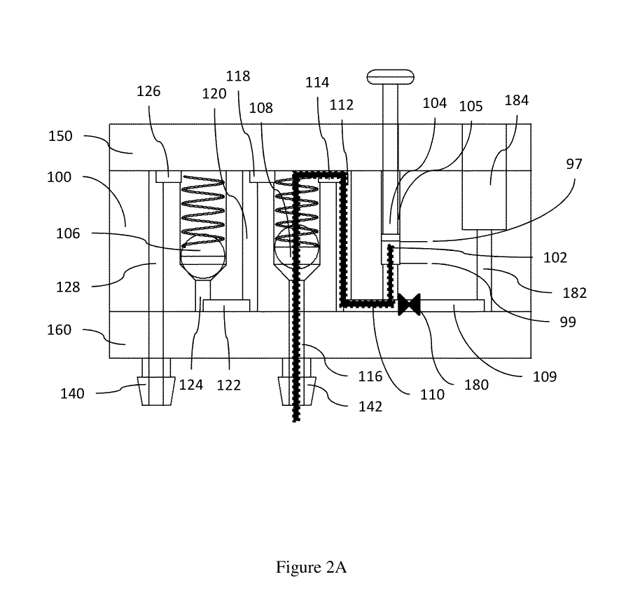

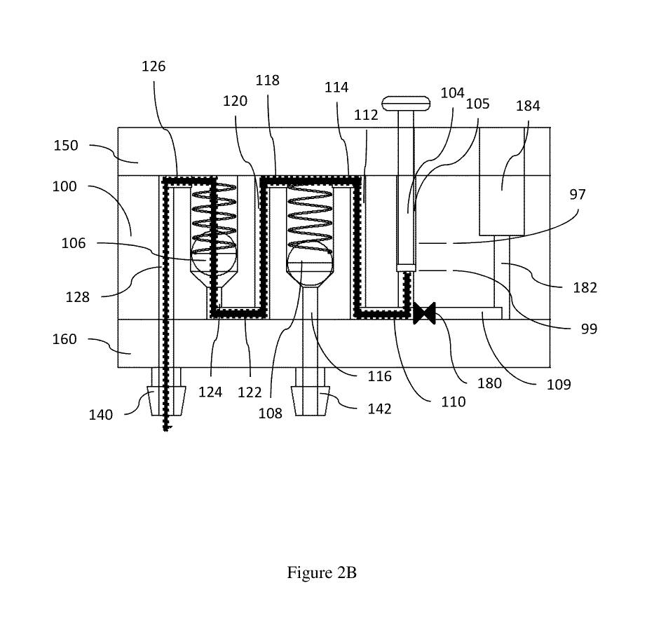

In an alternative embodiment as shown in FIGS. 2A and 2B, the check valves are in series with each other connected by a channel to the fluid displacement mechanism. In this embodiment, a fluid inlet 142, a fluid outlet 140, an outlet check valve 106, an inlet check valve 108, a moving member 104 and seal 32, a moving member holder 105, an isolation feature 180, and multiple connecting channels 128, 126, 124, 122, 120, 118, 116, 114, 112, 110, 109 and 182 of the device are shown in FIG. 2. Preferably, the moving member is disposed in the channel between the inlet check valve and the isolation feature, so that the isolation feature is downstream from the moving member during fluid filling of the channel. Fluid is manipulated in the device to achieve dispensing of a precise, pre-determined fluid volume. To accomplish this, moving member 104 displaces liquid in a cavity 102 by creating either a positive or negative pressure. The displacement occurs when the moving member 104 is alternated between the fill position stop 97, shown in FIG. 2A, and the dispense position stop 99, shown in FIG. 2B. The cavity is created and filled when the moving member 104 moves from the dispense position stop 99 to the fill position stop 97 and the fluid is displaced from the cavity when the moving member 104 moves from the fill position stop 97 to the dispense position stop 99. As shown in FIG. 2A, in one embodiment cavity 102 is connected to channels 110 and 109 by a channel segment. However, the dispense position stop can be flush or nearly flush with the top of channels 110 and 109 in other embodiments, which design results in a flow path for optimal sweeping out of bubbles in the channel. The moving member 104 along with outlet check valve 106 and inlet check valve 108 determines the particular flow path the fluid will take. The fill and dispense position stops may be controlled through as known in the art, for example, by physical stops or position sensors. Connecting channels may have various shapes, surfaces, and pathways depending on the desired fluidic path and behavior. Surfaces may be native or tuned or modified to be hydrophilic or hydrophobic or a mix in order to affect wetting properties of a given fluid. A bubble trap may be incorporated in the fluidic pathway if desired. Many check valve types and mechanisms may be employed as are known to those skilled in the art.

To prime, purge or ready the device for use, such as removing air from the system or inserting or replacing fluid media, according to FIG. 2A fluid is shown being drawn into the device through the fluid inlet 142 by movement of the moving member 104 to the fill position stop 97 creating a negative pressure which opens inlet check valve 108 while outlet check valve 106 remains closed. The fluid travels from the fluid inlet 142, through channel 116, and through the open inlet check valve 108. The moving member has set positions, a fill position stop 97 and a dispense position stop 99. The fluid travels to the cavity 102 and to an isolation feature 180, such as a membrane, hydrophobic break, or valve. Once the moving member 104 has reached the fill position stop 97 and fluid has filled the cavity 102, the isolation feature is opened. Now when the moving member 104 is moved to the dispense position stop 99, the fluid will move through channel 109, through the open isolation feature 180, through channel 182 and finally exit the device through purge feature 184. This sequence of steps, whereby the isolation feature 180 is in the closed position while the moving member 104 is moved to the fill position stop 97 and then the isolation feature 180 is changed to the open position while the moving member 104 is moved to the dispense position 99, may be repeated to prime the device from the fluid inlet 142 to the purge feature 184. Purge feature 184 may be a chamber in the device or may serve as a further exit orifice for the device. Following this purge the isolation feature 180 is closed and remains so during the remaining priming and operation. To prime the device from the fluid inlet check valve 108 to the fluid outlet 140, the moving member 104 is repeatedly moved between the fill position stop 97 and the dispense position stop 99. This priming sequence effectively removes any air or gas from the dispensing channels of the device, filling it completely with fluid.

An embodiment priming sequence could involve first opening the isolation feature 180, and then providing a flow from an external source with sufficient pressure and volume to fill portions of the device with fluid, and rid it of air or gas. Fluid from this external fluid source would enter the device through purge feature 184. Preferably, the moving member is in the dispense position stop flush with the chamber wall so no cavity is formed and fluid travels past the moving member to the fluid outlet. At this point air or gas has been removed from the system from the fluid outlet 140 to the isolation feature 180, and the device can be used for fluid manipulation. In an embodiment, the isolation feature can be open and fluid can be forced from the fluid inlet through the isolation feature. The entire device can be purged of air by alternating the moving member 104 repeatedly between the fill position stop 97 and the dispense position stop 99, which will cause fluid to travel through channels 114, 118, 120, 122, and 124, through check valve 106, through channels 126 and 128, and finally through the fluid outlet 140. Alternatively, to void the entire device of air, the isolation feature 180 can be closed. Displacing moving member 104 from the fill position stop to the dispense position stop forces open outlet check valve 106, closes inlet check valve 108, so as to fill channels 118, 120, 122 and 124, outlet check valve 106, channels 126 and 128, and the fluid outlet 140. At this point air or gas has been removed from the entire system.

Alternately, if the device or its fluidic components are under vacuum prior to filling with fluid, the device will readily aspirate the fluid, such as insulin. This would serve to prime the device, eliminating any air from the fluid path. The entire device or selected components of the device could be placed and stored under vacuum at the time of manufacture. Alternatively, the channels could be pre-evacuated or the vacuum accomplished at the time of use with a vacuum system method.

To operate the device shown in FIGS. 2A and 2B, the moving member 104 moves between the fill position stop 97 and the dispense position stop 99. The moving member preferably has a seal and can displace fluid by creating either a positive or negative pressure. In the configuration shown in FIGS. 2A and 2B, both check valves 106 and 108 are positioned in series in the channel between the inlet and outlet. The inlet check valve 108 is positioned in the channel between the fluid inlet 142 and the moving member 104 such that the open position allows fluid flow in the direction from the fluid inlet 142 to the cavity 102 and the closed position prevents fluid flow out the fluid inlet. The outlet check valve 106 is positioned in the channel between the fluid outlet 140 and the moving member 104 such that the open position allows fluid flow in the direction from the cavity 102 to the fluid outlet 140 and the closed position prevents fluid flow in the direction from the fluid outlet 140 to the inlet check valve 108. When moved to the fill position stop 97, the cavity 102 is created and generation of negative pressure causes fluid to move from the fluid inlet 142, through channel 116, through open inlet check valve 108, through channels 114, 112, and 110, to the nascent cavity 102, as shown in FIG. 2A. The fluid does not travel through channels 118, 120, 122 and 124 due to closed outlet check valve 106. Once the cavity 102 is filled, the fluid movement stops and the intake check valve closes. The moving member 104 is moved to the dispense position stop 99, creating a positive pressure. Now the volume of fluid that was in the cavity 102 travels through channels 110, 112, 114, 118, 120, 122, and 124, through open outlet check valve 106, through channels 126 and 128, and through the fluid outlet 140, as shown in FIG. 2B. When in the dispense mode, inlet check valve 108 is in the closed position so the fluid is forced to pass through open outlet check valve 106. The volume of fluid expelled from the fluid outlet 140, is equivalent to the volume of the cavity 102 created by the moving member 104. The volume of the cavity 102 can be adjusted to the desired metered aliquot volume.

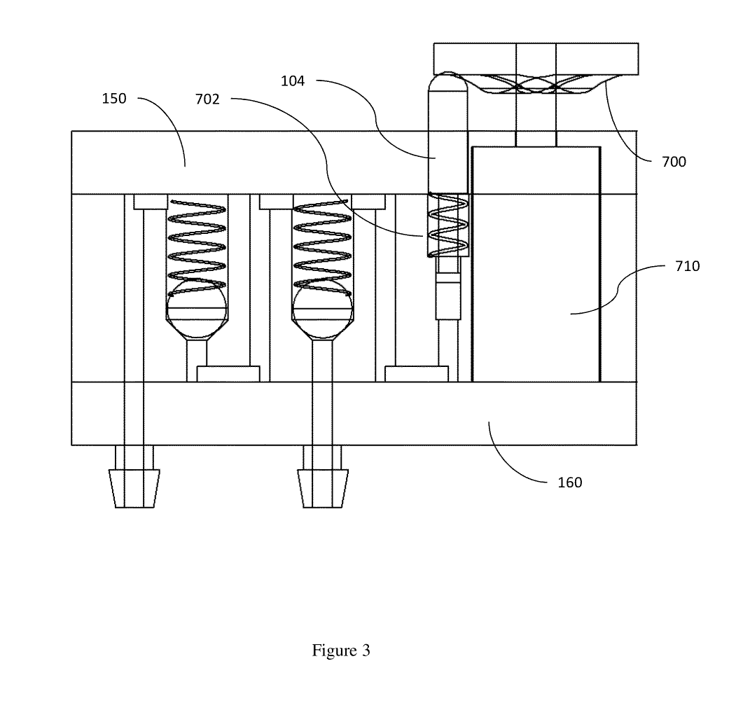

The movement between stops of the moving member 104 may also be limited by the drive components, such as lever travel or travel by the translation hardware or mechanism. An embodiment shown in FIG. 3 includes a rotational mechanism 700 wave structure that when rotated moves the member 104. A return force spring 702 returns member 104 to its first position. Repeated action moves the moving member in a reciprocal fashion. A double sided CAM or wave disk could actively return member 104 to an initial position. Other mechanisms and linkages would include various types of springs and materials capable of preloading. The movement is driven by a motor 710 or other suitable device. Translational mechanisms are known in the art for movement of the member between the first and second movement. Suitable mechanisms include, but are not limited to, lead screw, worm drive, levers, pinion, cam, gears, electromagnetic actuation, and the like. The drive force could be located in the main fluid block 100, in either of the end caps 150 or 160 or alternatively, external from the device itself.

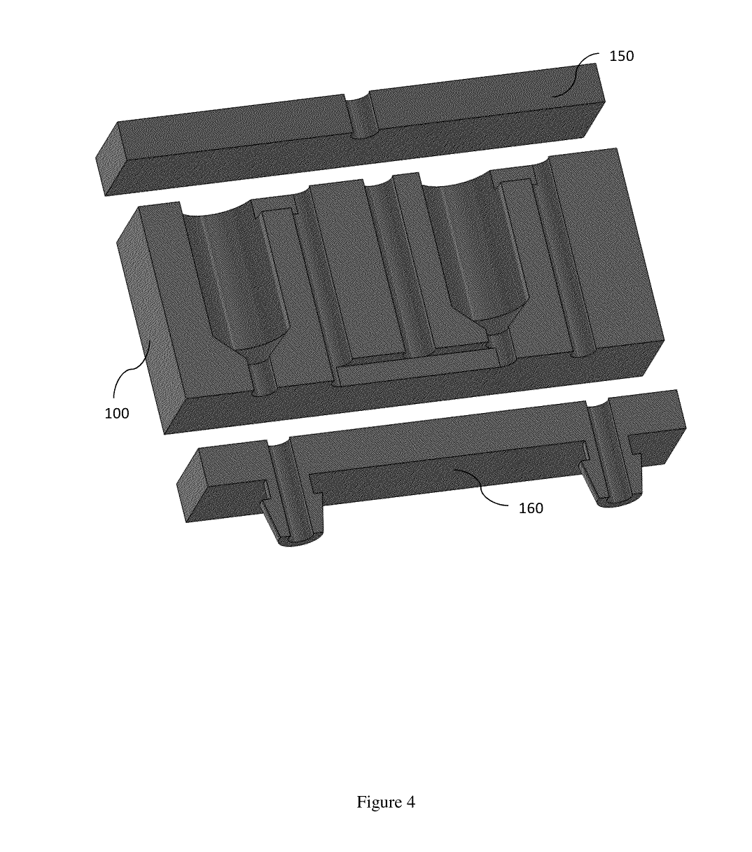

This invention allows for architectures for manufacturing the device that are readily amenable to injection molding. In this device the housing composed of primary pumping block 100, and end caps 150 and 160, as shown in FIG. 4 as a cross-section of one half of the device, is capable of accepting all major internal components, including sealing and moveable components, from the top plane of the primary block during assembly. The primary block 110, top and bottom end caps 150 and 160 are each mold releasable structures, suitable for injection molding. Connecting channels, such as 52, 62, and 56/58, run perpendicular at the outer surfaces of the primary block 100. The through structure features of the main fluid block 100 allow for precise injection molding and efficient part release from the mold cavity. The single plane orientation of the check valves and the serpentine nature of the connecting channel design allows for part integration in the primary block by dropping in from the top rather than connecting isolated components external from the main fluid path.

This device can be fabricated as an injection mold releasable main fluid block 100, with two end caps 150 and 160, as shown as a cross-section in FIG. 4. The end caps 150 and 160 can be attached to the main fluid block 100 after insertion of the internal components through mechanical connection, adhesion, bonding, welding (including ultrasonic and laser), fusing, melting, or the like. Additionally there may be another material between the end cap and the fluid block for connection and sealing such as, but not limited to, a gasket, o-ring, washer, or the like. Alternatively, sealing can be achieved through press tight or bonding features.

The connecting channels running perpendicular to the major components are formed when the end caps 150 and 160 are sealed to the main fluid block 100. Alternatively, the connecting channel features could be contained in the end caps 150 and 160, instead of in the main fluid block 100. The end caps 150 and 160 could also contain additional structures or features, such as channels or clearance holes. The end caps may also serve as an interface to other hardware or as a manifold. The end caps may have quick connects, threads, and press fit features. The end caps may also serve as a fluid reservoir.

In one embodiment, the channels or fluid paths that run through the block 100 from top to bottom, such as in FIG. 1A 50, 54, 105, 60, and 64, may be injected molded with taper angles ranging from 0-3 degrees. The tapers allow for efficient part release from the mold upon the plastic cooling. The mold cavity may be machined as one monolithic piece or consist of more than one piece with inserts. The mold may have all of the through features derive from the one side of the mold or in another embodiment the structures in the mold channel features may derive from both the A and B side of the injection mold. The sides of the mold are commonly referred to as the “cavity” (A-side) and the “core” (B-side). For example in FIG. 1A, channels 52 and 62 may be formed from one side of the mold and 56 and 58 from the other side of the mold. The layout is dependent on the final mold design and understood by those skilled in the art. The filling and gating of the plastic will be based on the end design of the mold. The surfaces of the mold may have finishes suitable for creating the fluidic sealing of the check valves and sealing member 104 and 32. However in another embodiment, inserts may be placed in the block if alternative materials are desired. The part may be molded with one type of plastic or over molding may be used if multiple materials are desired. In another embodiment, assemblies or preformed check valves may be inserted in the block channels. Secondary operations for improving bonding and adhesion of the surface may be implemented. Alternatively, the device may be machined in metal or plastic for low quantity production.

This invention also allows for facile device assembly. By having all major features in a single plane from the top of the block, all internal components can be inserted into the main fluidic block 100 from the top plane or surface. For assembly, the check valves 106 and 108 and the moving member 104 can be inserted on the top side, see FIG. 1. In one embodiment, check valves 106 and 108 and the moving member 104 could be dropped into the main fluidic block 100 and then end cap 150 could be attached to the main fluidic block 100. End cap 160 could be attached to the main fluidic block 100 either before or after the insertion of the components. Thus, the main fluid block and end caps when assembled forms the housing upon which the moving parts, such as the piston, check valve balls and springs can be inserted. In an embodiment, the moving parts can be inserted in a single plane in a block half prior to assembly of two block halves or can be dropped in to an assembled housing from above prior to attachment of the upper end cap.

This design allows the manufacturing of the invention by using automated or semi-automated assembly processes. The device allows for assembly via such systems as pick-and-place automation systems. Alternatively, the devices could be manually assembled with limited part reorientation.

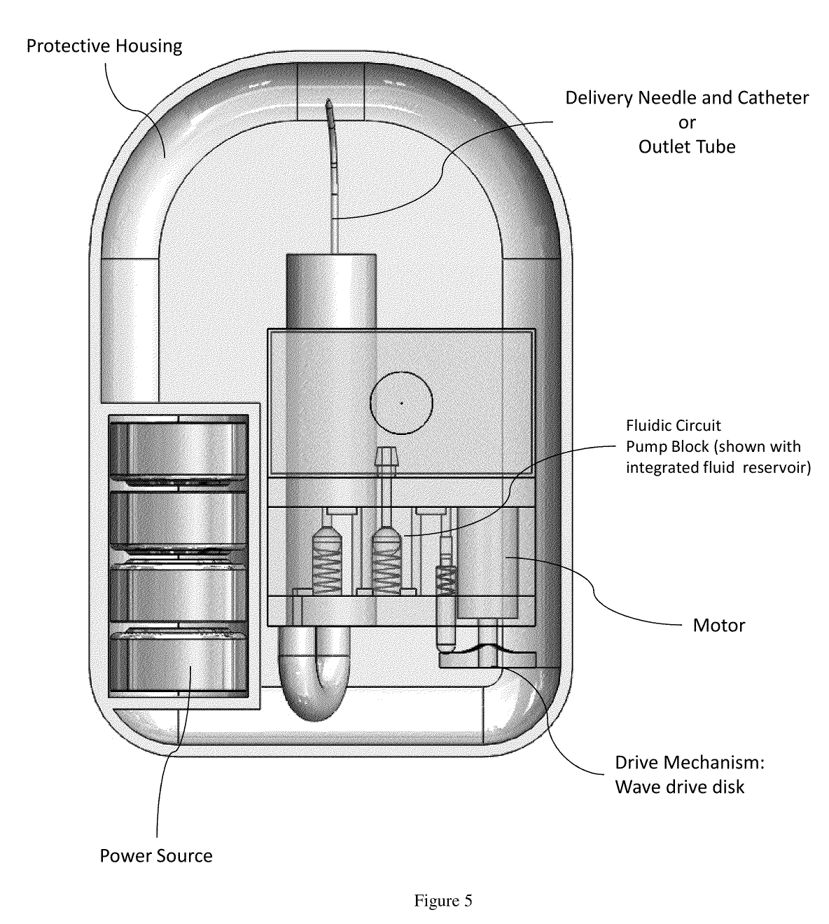

The invention can be implemented as a modular design where the device can contain components that can be re-used and then, when nearing expiration, can be easily replaced with new components by the end-user. Components that could be replaced in an embodiment include the power and drive system which is composed of a power source, mechanical force drive system, and control electronics. The invention could also employ replacement, pop-in components for other features of the system, such as the battery pack and all fluid touching or containing portions of the device including the fluid source reservoir, the main fluidic block with end caps, and the needle/catheter portion which interfaces with the user (FIG. 5). In another embodiment the needle/catheter could be any interface to a body or other device. The batteries or power source could be replaced after a period of use. Alternatively, the device could be fully disposable.

The system may have an energy source to power electronic and mechanical functions of the device which may include batteries, a wired energy source, harvested energy, such as vibration, MEMS harvested, mechanical movement, or pre-loaded mechanical forces, such as a spring, memory metal, compressed gas, and the like.

The invention could have integrated electronics running in an independent manner. Alternatively, the system could also have wireless communication to converse with other systems, controllers or signal receivers. Communication could occur through signals such as, but not limited to, Wifi, IR, Bluetooth, or radiofrequency.

A system incorporating the device is shown in FIG. 5. In an embodiment, the device is shown enclosed in a protective housing with a power source, a drive mechanism, a motor, and a delivery needle or catheter. In another embodiment the delivery needle or catheter could be replaced by other modes of interfacing the invention with a body or other device.

In a preferred embodiment, the device including housing and all drive and fluidic components can be less than about 3×3×0.5 inches and preferably less than about 1.5×2 inches or equivalent in surface area. The pump block dimensions preferably range from about 2×2×0.5 inches, or more preferably about 0.75×0.3×0.25 inches.

Preferably check valves, such as ball check valves, are equipped with springs to ensure a minimum pressure difference between the output back pressure and pressure which is generated by the moving member. The piston force is ˜equal to (back pressure+valve cracking pressure)*piston area+piston seal friction force. The system back pressure may be increased through the use of a restriction feature (tube or small aperture) or increased backpressure. The valve springs have the function of opening or closing the valve in response to the pressure difference between the valve's output side (system back pressure) and input side (piston). The type and dimensions of the spring as well as stiffness, and pre-load may be chosen based on the desired crack pressure for the system. For example, if higher cracking pressure is desired relative to a larger piston, a stronger spring (spring with a greater pre-load) may be used. This feature is similar to elastomer check valves and other type check valves.

The device pumping out force is in a range from about 0.01 PSI to about 100 PSI with preferred press ability in the range of from about 0.1 PSI to about 10 PSI. Flow rates in accordance with the present invention preferably range from nanoliter per minute to microliters per minute.

Due to the pump stroke generating a given volume per cycle, a pulsing profile will result from a reciprocating motion of the piston. The intervals in the pumping cycle (reciprocation of the piston) will result in a momentary decrease in fluid flow and system pressure. This “off time” is the interval when the piston has finished the dispense stroke and is starting the refill stroke. In a case where the pump is desired to be running continuously or near continuous, the pulse may be minimized by adding a restrictor or pulse dampener to the outlet stream of the pump. To maintain a more constant flow, a pulse compensator, or dampener, that stores energy during the pump's delivery stroke and returns an appropriate amount of work to the fluid during the pump's off time can be used. The pulse dampener will smooth flow pulsations and help maintain a more constant system pressure. The dampener reduces pulsations by compressing the fluid held within a chamber contained in the unit. This is accomplished by implementing a durable, but flexible, diaphragm to expand chamber volume. As system pressure increases during the pump delivery stroke, fluid in the chamber is compressed and the diaphragm expands. When the pump begins its refill stroke, the expanded diaphragm compresses the fluid, keeping the fluid flowing at a near constant rate and maintaining system pressure. Similarly, a restrictor or pulse dampener can be added to the inlet stream of the pump.

As explained herein, the device can both draw and expel fluid. Thus, the device can pump fluid through a location positioned downstream from the fluid outlet as well as draw fluid through a location positioned upstream from the fluid inlet. This design is advantageous when desiring to provide a fluid flow which operates under conditions, such as for example heating, cooling, or sterile, which can be separated from the internal pump components.

Further aspects of the present invention include the following. The ability to provide high precision injection, wherein volume is increased by increasing the number of injections/stroke cycles while precision is maintained constant since a fixed volume chamber is utilized. Accuracy and precision is determined by the volume of the defined cavity, wherein full displacement of the cavity contents equals high repeatability of the flow. This approach alleviates the precision typically required of a partial dispense, such as compared to where a syringe travel defines the volume dispensed. According to the present design, the moving components tolerance/movement does not need to be high/tight because there can be a stop or over travel to fill the dispense structure. The injector system may be independent of the mechanical drive system, such as in a partially disposable device, wherein a consumable portion of the device can be replaced without replacing the entire system. Alternately, the injector system may be integrated as part of the mechanical drive system, such as in a fully disposable system.

Although various embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A microfluidic delivery device for pumping a predetermined volume of fluid comprising:
a housing comprising a channel connecting a fluid inlet and a fluid outlet;
a moveable member positioned in the channel between the fluid inlet and the fluid outlet, moveable between a fill position stop and a dispense position stop, where a positive pressure is created in the channel when the member moves to the dispense position stop and negative pressure is created in the channel when the member moves to the fill position stop;
a cavity capable of accepting fluid from the channel when the moveable member moves from the dispense position stop to the fill position stop;
an inlet check valve positioned between the fluid inlet and the moveable member such that an inlet check valve open position allows fluid flow in the direction from the fluid inlet to the moveable member and an inlet check valve closed position prevents fluid flow in the direction from the moveable member to the fluid inlet; and
an outlet check valve positioned between the fluid outlet and the moveable member such that an outlet check valve open position allows fluid flow in the direction from the moveable member to the fluid outlet and an outlet check valve closed position prevents fluid flow in the direction from the fluid outlet to the moveable member,
wherein the outlet check valve is in the closed position when fluid is drawn from the fluid inlet and through the open inlet check valve into the cavity when the moveable member is moved to the fill position stop and the inlet check valve is in the closed position when fluid is expelled from the cavity through the open outlet check valve and out the fluid outlet when the moveable member is moved to the dispense position stop.

2. The device of claim 1, further comprising a drive force system sufficient to move the member from a first position to a second position when the member is acted on by the drive mechanism.

3. The device of claim 1, further comprising a fluid reservoir in communication with the fluid inlet.

4. The device of claim 1, wherein the cavity volume is adjustable.

5. The device of claim 1, wherein the fluid expelled from the fluid outlet is administered to a person.

6. The device of claim 1, wherein the fluid outlet passes through a pulse dampening device.

7. The device of claim 1, wherein the fluid expelled from the fluid outlet supplies a fluidic device.

8. A microfluidic delivery device for pumping a predetermined volume of fluid comprising:
a housing comprising a channel connecting a fluid inlet and a fluid outlet;
a moveable member in communication with the channel between the fluid inlet and the fluid outlet, moveable between a fill position stop and a dispense position stop, where a positive pressure is created in the channel when the member moves to the dispense position stop and a negative pressure is created in the channel when the member moves to the fill position stop;
a cavity capable of accepting fluid from the channel when the moveable member moves from the dispense position stop to the fill position stop;
an inlet check valve positioned in the channel between the fluid inlet and the moveable member such that an inlet check valve open position allows fluid flow in the direction from the fluid inlet to the cavity and an inlet check valve closed position prevents fluid flow out the fluid inlet;
an outlet check valve positioned in the channel between the fluid outlet and the movable member such that an outlet check valve open position allows fluid flow in the direction from the cavity to the fluid outlet and an outlet check valve closed position prevents fluid flow in the direction from the fluid outlet to the inlet check valve; and
an isolation feature in communication with the cavity,
wherein the outlet check valve is in the closed position when fluid is drawn from the fluid inlet and through the open inlet check valve into the cavity when the moveable member is moved to the fill position stop and the inlet check valve is in the closed position when fluid is expelled from the cavity through the open outlet check valve and out the fluid outlet when the moveable member is moved to the dispense position stop.

9. The device of claim 8, further comprising a drive force system sufficient to move the member from a first position to a second position when the member is acted on by the drive mechanism.

10. The device of claim 8, further comprising a fluid reservoir in communication with the fluid inlet.

11. The device of claim 8, wherein the cavity volume is adjustable.

12. The device of claim 8, wherein the fluid expelled from the fluid outlet is administered to a person.

13. The device of claim 8, wherein the fluid outlet passes through a pulse dampening device.

14. The device of claim 8, wherein the fluid expelled from the fluid outlet supplies a fluidic device.

15. The device of claim 1, wherein the housing is capable of being manufactured by injection molding.
